(19) United States
(12) Patent Application Publication
(10) Pub. No.: US 2015/0174070 A1
(43) Pub. Date: Jun. 25, 2015

(54) LIPOSOME SUSPENSIONS, METHOD FOR PREPARING THE SAME, AND APPLICATION THEREOF

(71) Applicant: Pharmosa Limited, Taichung (TW)

(72) Inventors: Mei-Ling Cheng, Taichung (TW); Yao-Kun Huang, Taichung (TW)

(21) Appl. No.: 14/578,830

(22) Filed: Dec. 22, 2014

(30) Foreign Application Priority Data
Dec. 24, 2013 (TW) 102147911

(51) Int. Cl. A61K 9/127; A61K 31/704

(52) U.S. Cl. CPC A61K 9/1277; A61K 9/127; A61K 31/704

(57) ABSTRACT

A method for preparing the liposome suspensions comprising liposomes with small particle size and uniform particle size distribution by performing an injection process in combination with a one-step extrusion, and further the liposome suspensions obtainable by this method as well as drug-encapsulating liposomes as well as a system for preparing the said liposome suspensions are disclosed.

### BACKGROUND OF THE INVENTION

1. Field of the Invention

The present invention relates to a method for preparing liposome suspensions, especially to a method for reducing particle size, narrowing particle size distribution, and large-scale production of the liposome suspensions. The present invention also relates to a liposome suspension prepared by the method, wherein an average particle size of the liposomes in the suspension is from 10 nm to 200 nm, and polydispersity index (PDI) is from 0.01 to 0.5. The present invention also relates to a method for encapsulating a drug with the liposome suspension, and the liposome suspension comprising the drug-encapsulated liposomes prepared by the method.

2. Description of the Prior Art

A liposome is a micro-closed vesicle that has an internal aqueous phase enclosed by at least one bilayer membrane per vesicle; within a liposome, hydrophilic materials are trapped in the internal aqueous phase while lipophilic materials are trapped in the lipid bilayer. The liposome can be a carrier of drugs, chemical compounds, and genetic materials, and it can protect materials from destruction by enzymes in human body by encapsulating them. The materials encapsulated within the liposome can be released in specific locations for drug delivery or therapeutic purposes. Clinical studies reveal that targeted therapy can be achieved by using unilamellar vesicles (UVs) of the liposome to encapsulate and deliver the drugs to tumor or liver cells specifically.

Conventional methods for preparing the liposome comprise hydration, ultrasonification, reverse-phase evaporation, surfactant treatment, pore extrusion, high-pressure homogenization, etc. U.S. Pat. No. 6,596,305 reveals that liposome suspensions can be obtained by first dissolving lipids in a water soluble organic solvent to form a mixture, and then the mixture is directly added to an aqueous solution and stirred. The concentration of lipid solution prepared according to the U.S. Pat. No. 6,596,305 is from 0.03 mg/ml to 0.8 mg/ml, which is too low to be used for large-scale production, and the stirring procedure is operated at a rotational speed that is too high (2,000 rpm). In addition, the concentration of the organic solvent in said solvent system needs to be adjusted repeatedly to screen for the most proper particle size of the population of liposome. The screening process is complex, and the obtained liposome is large, with an average particle size of 200 nm to 300 nm. Disadvantages described above, such as the high rotational speed and the complex screening process, are against large-scale production.

U.S. Pat. No. 5,000,887 proposes that after a lipid solution is obtained by dissolving lipids in a water-soluble organic solvent (such as ethanol), an aqueous phase is added into the lipid solution slowly to form lipid suspensions. Then the water-soluble organic solvent is removed from the lipid suspensions by reverse osmosis or evaporation to raise a ratio of water to water-soluble organic solvent. Although the particle size of the liposome prepared according to the U.S. Pat. No. 5,000,887 is less than or equal to 300 nm, the complexity of the above methods such as the continuous removal of the water-soluble organic solvent makes them inapplicable for large-scale production.

U.S. Pat. No. 4,687,661 proposes using a water-soluble and non-volatile organic solvent (such as polyhydric alcohols, glycerin esters and benzyl alcohol) to dissolve lipids for preparing a lipid solution. The lipid solution is added into an aqueous phase directly and stirred to form a liposome suspension. The particle size of the liposome prepared according to the U.S. Pat. No. 4,687,661 depends on the type of mixer employed. Smaller particle size of the liposome is obtained by strong or high frequency stirring of the lipid solution. For example, larger particle size of the liposome is achieved by stirring the lipid solution with a propeller mixer; smaller particle size of the liposome is achieved by stirring the lipid solution with a high shearing method (such as homogenizer), and even smaller particle size of the liposome is achieved by stirring the lipid solution with ultrasonics or high pressure homogenizer methods. The non-volatile organic solvent used in the preparation methods of the U.S. Pat. No. 4,687,661 is non-toxic; however, the lipid must be dissolved or hydrated at a high temperature (higher than 90° C.). Besides, poor solubility of lipids in the water-soluble and non-volatile organic solvent leads to the formation of liposomes with larger particle size (about 500 nm to several μm) and a wider range of particle size distribution. For clinical use, the liposomes need to be further processed to reduce the particle size and to achieve a uniform particle size distribution. The above methods of the U.S. Pat. No. 4,687,661 are time-consuming and result in poor liposome quality. Therefore, the methods of the U.S. Pat. No. 4,687,661 are not suitable for large-scale production of liposomes.

U.S. Pat. No. 5,077,057 suggests using a mixed solvent made up of an aprotic solvent and lower alkanols to dissolve drugs and lipids for preparing a lipid solution. The lipid solution with drugs is injected into an aqueous solution at a speed of 0.5 ml/min to 10 ml/min accompanied with high speed stirring of 250 rpm to 750 rpm to form liposome suspensions. The injection rate used in this method is too slow, the resulting range of particle size distribution is too wide, and the mixed solvent that includes dimethyl sulfoxide (DMSO), dimethylformamide (DMF), or dimethylamine (DMA) is too toxic to human body and not suitable for clinical application. Besides, the manufacturing process is time-consuming and inapplicable for large-scale production.

U.S. Pat. No. 5,008,050 suggests using chloroform to dissolve lipids for preparing a lipid solution. A lipid film is obtained by evaporating the chloroform from the lipid solution. An aqueous solution is then added into the lipid film for hydration to form multilamellar vesicles (MLVs), and the multilamellar vesicles are extruded through a filter apparatus comprising two stacked polycarbonate filter membranes. The pore size of the polycarbonate filter membranes determines the particle size of the liposome; besides, to extrude the MLVs through the polycarbonate filter membranes without filter clogging, a pressure between 100 psi and 700 psi must be applied in order to reach a flow rate on the order of 20 ml/min to 60 ml/min. Because operating under high pressure is relatively dangerous, the filter apparatus is complex, the MLVs need to be prepared first to produce small unilamellar vesicles (SUVs), and the procedures performed above are time-consuming, the manufacturing process of the U.S. Pat. No. 5,008,050 is not suitable for large-scale production.

Taiwan patent No. I391149 also suggests using chloroform to dissolve lipids for preparing a lipid solution. A lipid film is obtained by evaporating the chloroform from the lipid solution. An aqueous substrate is added into the lipid film at a temperature between 71° C. and 86° C. to form MLVs. In order to reduce the particle size of liposomes, large unilamellar vesicles (LUVs) need to be prepared first by applying the MLVs to freeze-thaw or sonication procedures followed by an extrusion method. The extrusion method works by extruding the LUVs vesicles through three polycarbonate filter membranes of decreasing pore sizes: from 200 nm to 100 nm and finally to 50 nm to obtain SUVs. Because MLVs need to be made at a high temperature and processed to obtain LUVs, and then to go through multiple extrusion steps to form SUVs, the manufacturing process of the Taiwan patent No. I391149 is too complex and time-consuming for large-scale production.

Taiwan patent No. I250877 proposes using alcoholic solvent to dissolve lipids for preparing a lipid solution. The lipid solution is added into an aqueous solution directly to form lipid suspensions. The lipid suspensions are extruded through a filter membrane with a pore size of 100 nm at a pressure between 40 psi and 140 psi for 10 times, followed by extruding the lipid suspensions through another filter membrane with a pore size of 50 nm at a pressure between 40 psi and 140 psi for 10 times to obtain a filtrate. The filtrate is dialyzed by sucrose aqueous solution. Because the lipid solution forms MLVs of larger particle size, the MLVs need to be processed under higher pressure by a two-step extrusion method, where the MLVs go through two filter membranes of different pore sizes to obtain LUVs or SUVs. The manufacturing cost of Taiwan patent No. I250877 is too high for large-scale production.

Due to the aforementioned shortcomings of conventional techniques for preparing liposomes, such as complex procedures, high-pressure or high-temperature operation, and extrusion through filter membranes with different pore sizes for multiple times, the conventional techniques are too costly and time-consuming for large-scale production.

The present invention overcomes the aforementioned shortcomings and provides a method for preparing lipid suspensions that is efficient, cost-effective, and suitable for large-scale production.

### SUMMARY OF THE INVENTION

The main objective of the invention is to provide a method for industrial preparation of liposome suspensions. The method comprises (1) setting process parameters of injection flow rate to an apparatus to obtain unilamellar vesicles (UVs) and (2) simple preparation procedures with a filter membrane of a single pore size to produce liposome suspensions having small particle size and narrow particle size distribution range for clinical use and large-scale production.

The method for preparing liposome suspensions in accordance with the present invention comprises providing a component that comprises phospholipid, cholesterol or cholesterol salt derivatives, and polyethylene glycol derivatives. The molar ratio of the phospholipid to the cholesterol or the cholesterol salt derivatives to the polyethylene glycol derivatives is 3-50:1-50:1.

The component is mixed with an alcoholic solvent to form a mixture, whose concentration ranges from 2 mM to 300 mM. The mixture is then injected into an aqueous solution under thermal condition by an injection apparatus followed by stirring to form the liposome suspensions. The volume ratio of the mixture to the aqueous solution is between 1:2 and 1:500.

In a preferred embodiment, the volume ratio of the phospholipid to the cholesterol or the cholesterol salt derivatives to the polyethylene glycol derivatives of the component is 4-20:2-10:1.

In a preferred embodiment, the alcoholic solvent is lower alkanols.

The lower alkanols in accordance with the present invention comprise, but are not limited to, methanol, ethanol, propanol, isopropanol, butanol, and acetone.

More preferably, the alcoholic solvent is ethanol.

It is preferred that the phospholipid of the component is selected from the group consisting of lecithin, phosphatidylcholine (PC), phosphatidylethanolamines (PE), phosphoglyceride (PG), phosphatidylinositols (PI), phosphatidic acids (PA), and diacyl derivatives (C₁₂-C₂₂) thereof.

It is preferred that the cholesterol or the cholesterol salt derivatives are selected from the group consisting of cholesterol sulfate, cholesterol hemisuccinate, and cholesterol phosphate.

It is preferred that the polyethylene glycol derivatives of the component are selected from the group consisting of polyethylene glycol-phosphatidyl ethanolamine (PEG-PE), [methoxy-poly(ethylene glycol)-phosphatidyl ethanolamine (mPEG-PE), and diacyl derivatives (C₁₂-C₂₂) thereof.

The injection apparatus in accordance with the present invention refers to an injection apparatus with controllable flow rate. The apparatus comprises at least one injection channel and a propulsion unit for flow rate control. The hole size of the at least one injection channel is less than 10 mm, and the at least one injection channel comprises one single hole or multiple holes. The propulsion unit comprises, but is not limited to, syringe pump, tubing pump, reciprocating pump, gas propulsion unit, and other propulsion units.

In a preferred embodiment, the thermal condition is between 40° C. and 80° C.

In a preferred embodiment, the aqueous solution is an ion solution, whose concentration is between 1 mM and 1 M. According to this embodiment, the ion solution is selected from the group consisting of sodium chloride, polyacrylate, chondroitin sulfate A, polyvinylsulfate, phosphate, pyrophosphate, sulfate, citrate, tartarate, nitrilotiacetate, ethylenediamine tetraacetate, diethylenetriamine pentaacetate, and their salt derivatives thereof.

Preferably, the ion solution is sulfate.

More preferably, the sulfate is ammonium sulfate.

It is preferred that the volume ratio of the mixture to the aqueous solution is between 1:2 and 1:100.

The stirring referred to in the step of “injecting the mixture into an aqueous solution under thermal condition by an injection apparatus followed by stirring to form the liposome suspensions” means to be mixed by mixers comprising but not limited to a magnetic stirrer, propeller mixer, homogenizer, and other type of mixers.

It is preferred that the stirring speed is between 100 rpm and 500 rpm.

It is preferred that the injection flow rate of the injection apparatus in the step of “injecting the mixture into an aqueous solution under thermal condition by an injection apparatus” is from 10 ml/min to 1000 ml/min.

More preferably, the injection flow rate of the injection apparatus is from 25 ml/min to 600 ml/min.

The invention further provides a method for preparing the liposome suspensions as described above, which further comprises an extrusion step for extruding the liposome suspensions through the filter membrane with pore size less than 100 nm.

It is preferred that the extrusion step comprises extruding the liposome suspensions through the filter membrane with pore size between 10 nm and 80 nm.

It is preferred that the pressure of the extrusion step is between 30 psi and 80 psi.

It is preferred that the flow rate of the extrusion step is between 2 L/min and 10 L/min.

Furthermore, the invention provides a liposome suspension, where the liposome particle size of the liposome suspension is between 10 nm and 200 nm, and the polydispersity index (PDI) of the particle size is between 0.01 and 0.5.

Preferably, the liposome particle size is between 30 nm and 120 nm, and the PDI is between 0.03 and 0.25.

The invention further provides a method for encapsulating drugs into the liposomes. The method comprises the following steps:
(1) preparing the drug;
(2) removing the alcoholic solvent of the liposome suspension by dialysis;
(3) mixing the drug and the liposome suspension to allow the drug to be encapsulated in the liposomes.

Preferably, the drug is selected from the group consisting of doxorubicin HCl, daunorubicin, gemcitabine, oxamniquine, fluconazole, itraconazole, ketoconazole, micronazole, irinotecan, and vinorelbine.

The invention further provides a liposome suspension comprising the drug-encapsulated unilamellar vesicles (UVs) prepared by the encapsulating method described above. The average particle size of the drug-encapsulated UVs of the liposome suspension is less than 200 nm, and encapsulation ratio of the UVs is higher than 95%.

The invention further provides a system for preparing the liposome suspensions. The system comprises a mixing chamber, an aqueous solution chamber and an injection apparatus located between the mixing chamber and the aqueous solution. The injection apparatus connects to the mixing chamber by a first channel, and the injection apparatus further comprises an injection channel and a first propulsion unit. The injection channel adjacent to the aqueous solution chamber connects to the one end of the first channel opposite to the mixing chamber connecting with. The injection channel comprises one single hole or multiple holes. The first propulsion unit is embedded in the first channel and located between the mixing chamber and the injection channel for pushing the solution in the mixing chamber to enter the aqueous solution chamber through the first channel and the injection channel. The aqueous solution chamber comprises a stirring unit and a heat maintaining unit, wherein the stirring unit is in the aqueous solution chamber, and the heat maintaining unit is adjacent to the aqueous solution chamber to maintain the temperature of the aqueous solution chamber.

In one embodiment, the first propulsion unit of the injection apparatus comprises, but is not limited to, syringe pump, peristaltic pump, piston pump, diaphragm pump, pneumatic unit, and other propulsion units.

In one embodiment, the stirring unit of the aqueous solution chamber comprises, but is not limited to, magnetic stirrer, propeller, homogenizer, and other stirring designs.

In a preferred embodiment, the hole size of the injection channel of the injection apparatus is less than 10 mm.

In a preferred embodiment, the flow rate of the injection apparatus is between 10 ml/min and 1000 ml/min.

More preferably, the flow rate of the injection apparatus is between 25 ml/min and 600 ml/min.

In a preferred embodiment, the heat maintaining unit maintains the temperature of the aqueous solution chamber between 40° C. and 80° C.

In a preferred embodiment, the system further comprises an extrusion apparatus, which connects to the aqueous solution chamber via a second channel. The extrusion apparatus comprises an extrusion unit, a second propulsion unit, a third channel, and a third propulsion unit. The second channel is connected to the aqueous solution chamber, and the extrusion unit is connected to the second channel opposite to the aqueous solution chamber connecting with.

The extrusion apparatus further comprises a first filter membrane. The second propulsion unit is embedded in the second channel, and is located between the aqueous solution chamber and the extrusion apparatus. Each of two terminal ends of the third channel is connected to each of two ends of the extrusion unit, respectively, forming a circulation loop. The third propulsion unit is embedded in the third channel to enhance circulation of the circulation loop between the extrusion unit and the third channel.

It is preferred that the pore size of the first filter membrane of the extrusion unit is less than 100 nm.

More preferably, the pore size of the first filter membrane of the extrusion unit is between 10 nm and 80 nm.

It is preferred that the pressure applied on the second propulsion unit is between 30 psi and 80 psi.

It is preferred that the flow rate of the extrusion unit is between 2 L/min and 10 L/min.

In one embodiment, the second propulsion unit or the third propulsion unit of the extrusion unit comprises, but is not limited to, syringe pump, peristaltic pump, piston pump, diaphragm pump, pneumatic unit, and other propulsion units.

In a preferred embodiment, the system further comprises a drug encapsulating apparatus. The drug encapsulating apparatus is connected to the extrusion unit via a fourth channel. The drug encapsulating apparatus comprises a dialyzer, a drug encapsulating chamber, and a fifth channel connecting the dialyzer and the drug encapsulating chamber. The dialyzer is connected to one end of the fourth channel opposite to the extrusion unit connecting with. The drug encapsulating chamber is connected to the dialyzer via the fifth channel.

In a preferred embodiment, the system furthermore comprises a filtration apparatus. The filtration apparatus is connected to the drug encapsulating apparatus via a sixth channel. The filtration apparatus comprises a filter, wherein the filter is connected to the drug encapsulating chamber via the sixth channel. The filter further comprises a second filter membrane.

It is preferred that the pore size of the second filter membrane is 200 nm.

In a preferred embodiment, the system furthermore comprises a collection apparatus, wherein the collection apparatus connects to the filtration apparatus via a seventh channel. The collection apparatus comprises a collector, wherein the collector is connected to the filter of the filtration apparatus via the seventh channel.

The invention provides a method to control the particle size of the liposome to less than 200 nm by adjusting specific parameters of the injection apparatus. Owing to that small particle size of the UVs has been prepared by using the injection apparatus at first step, the followed extrusion step does not require to operate at high pressure and can retain high flow rate. Therefore, the extrusion performance is enhanced greatly and useful for large scale operation. Furthermore, the extrusion step can efficiently narrow the particle size distribution of the liposome with a single pore size membrane. Compared to the shortcomings of conventional techniques comprising complex preparation processes, extreme operation condition such as high temperature and high pressure, low product quality, low productive efficiency, and high cost and high time consumption, the present invention shows many advantages including a relatively easy, time-saving, cost-saving, appropriate operation condition, and applicability for industrial production.

Other objectives, advantages and novel features of the invention will become more apparent from the following detailed description when taken in conjunction with the accompanying drawings.

### BRIEF DESCRIPTION OF THE DRAWING

FIG. 1 is a flow chat of the methods for preparing liposome suspensions in accordance with the present invention.

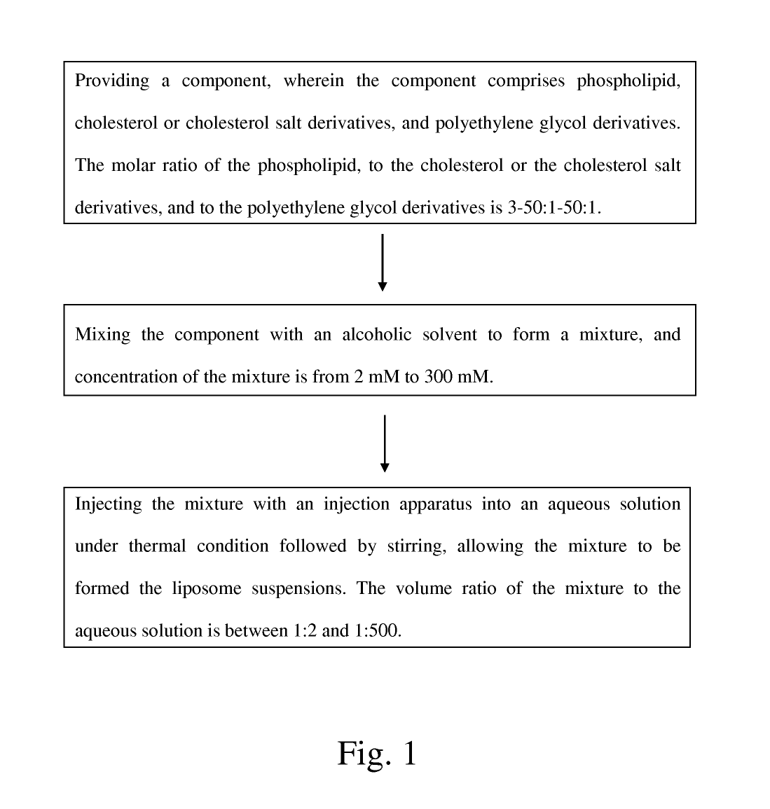

### DETAILED DESCRIPTION OF THE PREFERRED EMBODIMENTS

### Embodiment 1

Influence of injection flow rate on the particle size of liposomes.

33 g of ammonium sulfate was dissolved in water. And then the mixture was diluted to 1 L with water to form an ammonium sulfate solution followed by heating to 60° C. for use.

A homogeneous mixture of lipids was prepared by dissolving 4.8 g hydrogenated soybean phosphatidylcholine (HSPC), 1.6 g methoxypolyethylene glycol 2000 (MPEG-DSPE 2000), and 1.6 g cholesterol in 75 ml ethanol at 60° C.

Subsequently, the homogeneous mixture was injected into the ammonium sulfate solution with an injection apparatus, and kept stirring with a magnet at 200 rpm at 60° C. to obtain a liposome suspension. The injection flow rate was controlled at 25 ml/min, 100 ml/min, 150 ml/min, 200 ml/min, 250 ml/min or 300 ml/min by using peristaltic pumps.

The particle size of the liposomes obtained above was analyzed by the particle size analyzer, Delsa™Nano (Beckman Coulter, Inc).

TABLE 1
Effect of the injection rate on the particle size of the liposomes

| Injection flow rate (ml/min) | Particle size (nm) |
|---|---|
| 25 | 200 |
| 100 | 157 |
| 150 | 125 |
| 200 | 109 |
| 250 | 100 |
| 300 | 90 |

The data as shown in Table 1 presents that higher injection flow rate results in smaller particle size of the liposomes.

### Embodiment 2

Embodiment 2 relates to scale-up test.

495 g ammonium sulfate was dissolved in water, and then the mixture was diluted to 15 L with water to form an ammonium sulfate solution followed by heating to 60° C. for use.

A homogeneous mixture of lipids was prepared by dissolving 57.5 g HSPC, 19.2 g MPEG-DSPE 2000, and 19.2 g cholesterol in 1000 ml ethanol at 60° C.

Subsequently, the obtained homogeneous mixture of lipids was injected into the ammonium sulfate solution at the rate of 300 ml/min with the multi-hole injection apparatus, and kept stirring at 150 rpm in the propeller mixer at 60° C. to obtain a liposome suspension.

The particle size of the liposomes was analyzed by the particle size analyzer.

Results reveal that an average particle size of the obtained liposomes is 91 nm, and the polydispersity index (PDI) is 0.18.

### Embodiment 3

Embodiment 3 relates to extrude the liposome suspension by a single pore size of one-step extrusion.

The liposome suspension prepared by embodiment 2 was extruded through an extrusion apparatus adopting with a 50 nm polycarbonate filter membrane and connecting with two 20-L pressure vessels for extrusion process. During the extrusion process, the operating pressure was between 40 psi and 60 psi, and the flow rate was between 2 L/min and 10 L/min. Extrusion process was performed for 10 to 30 times repeatedly to achieve the desired particle size and size distribution of the liposomes.

The final liposome suspension was analyzed for the particle size of the liposomes with the particle size analyzer.

Results reveal that an average particle size of the liposomes is 80 nm, and the PDI is 0.07.

### Embodiment 4

Embodiment 4 relates to two-step extrusion.

The liposome suspension was prepared under the same condition as embodiments 2 and 3, wherein the extrusion process of the liposome suspension was divided into two stages.

After injection process, the liposome suspension was extruded through a 100 nm polycarbonate filter membrane at a pressure between 40 psi and 60 psi, and repeated it ten times. And then, the obtained liposome suspension was extruded another ten times through a 50 nm polycarbonate filter membrane.

The final liposome suspension was analyzed for the particle size of the liposomes with the particle size analyzer.

Results reveal that an average particle size of the extruded liposomes is 85 nm, and the PDI is 0.09.

### Embodiment 5

Embodiment 5 relates to two-step extrusion without the injection process.

33 g ammonium sulfate was dissolved in water, and then the mixture was diluted to 1 L to form an ammonium sulfate solution followed by heating to 60° C. for use.

A homogeneous mixture of lipids was prepared by dissolving 4.8 g hydrogenated soybean phosphatidylcholine (HSPC), 1.6 g methoxypolyethylene glycol 2000 (MPEG-DSPE 2000), and 1.6 g cholesterol in 75 ml ethanol with stirring at 60° C.

Subsequently, the obtained homogeneous mixture was added into the ammonium sulfate solution directly, and kept stirring to form a liposome suspension.

The liposome suspension was first extruded ten times through a 100 nm polycarbonate filter membrane at a pressure between 60 psi and 90 psi. And then, the obtained liposome suspension was extruded another ten times through a 50 nm polycarbonate filter membrane.

The final liposome suspension was analyzed for the particle size of the liposomes with the particle size analyzer.

Results reveal that an average particle size of the liposomes is 115 nm, and the PDI is 0.11.

The comparison of the particle size, the PDI, and the extrusion pressure between that of embodiment 2 (preparing by the injection process), embodiment 3 (preparing by the injection process and the one-step extrusion), embodiment 4 (preparing by the injection process and the two-step extrusion), and the embodiment 5 (preparing by the two-step extrusion) was shown in Table 2.

TABLE 2
Comparison of the particle size and size distribution between that of
the different preparation methods

| | | | Extrusion |
|---|---|---|---|
| | Particle | Polydispersity | pressure |
| Preparation methods | size (nm) | index, PDI | (psi) |
| Embodiment 2 | 91 | 0.18 | — |
| (injection process) | | | |
| Embodiment 3 | 80 | 0.07 | 40-60 |
| (injection process and one-step | | | |
| extrusion) | | | |
| Embodiment 4 | 85 | 0.09 | 40-60 |
| (injection process and two-step | | | |
| extrusion) | | | |
| Embodiment 5 | 115 | 0.11 | 60-90 |
| (two-step extrusion) | | | |

Liposomes of small particle size (less than 100 nm) with uniform particle size distribution were obtained by using the injection apparatus. More preferably, the particle size of the liposomes became more uniform and presented narrower distribution by further applying the single pore size extrusion. Because the UVs were obtained previously during the injection process, the following extrusion procedure could be operated under a lower pressure and extruded at a higher flow rate for producing a greater quantity and higher quality of the liposomes in the same time as compared with the conventional techniques. The liposome suspensions produced by the methods in accordance with the present invention were suitable for clinical use and large-scale production.

### Embodiment 6

Embodiment 6 relates to the preparation of a liposome suspension comprising drug-encapsulated UVs.

The liposome suspension treated by the extrusion process described in embodiment 3 was dialyzed at room temperature with 45 L of 9 wt % sucrose solution for substituting the ethanol and ammonium sulfate in the liposome suspension to obtain liposomes comprising ammonium sulfate inside and suspending in the sucrose solution. Then, 4.5 L of the dialyzed liposome suspension was collected for use.

18.9 g histidine was dissolved in 9 wt % sucrose solution to form a histidine solution, and the histidine solution was diluted to 450 ml for use.

12.0 g doxorubicin HCl was added into the dialyzed liposome suspension prepared previously followed by adding the histidine solution into the liposome suspension. The obtained drug-encapsulated liposome suspension was cooled to room temperature with a heat exchanger apparatus to accomplish the drug encapsulation of liposomes.

The drug-encapsulated liposome suspension was diluted with 9 wt % sucrose solution to 6 L and then preformed with sterile filtration. The suspension was then dispensed into sterile vials to be the final products which comprising 2 mg/ml doxorubicin HCl in each of the sterile vials.

Even though numerous characteristics and advantages of the present invention have been set forth in the foregoing description, together with the details of the structure and features of the invention, the disclosure is illustrative only. Changes may be made in the details, especially in the matters of shape, size, and arrangement of parts within the principles of the invention to the full extent indicated by the broad general meaning of the terms in which the appended claims are expressed.

What is claimed is:

1. A method for preparing liposome suspensions comprising:
(1) providing a component comprising phospholipid, cholesterol or cholesterol salt derivatives, and polyethylene glycol derivatives, wherein the molar ratio of the phospholipid to the cholesterol or the cholesterol salt derivatives to the polyethylene glycol derivatives is 3-50:1-50:1;
(2) mixing the component with an alcoholic solvent to form a mixture, wherein concentration of the mixture is between 2 mM and 300 mM; and
(3) injecting the mixture into an aqueous solution under thermal condition with an injection apparatus followed by stirring to form the liposome suspensions, wherein the volume ratio of the mixture to the aqueous solution is between 1:2 and 1:500.

2. The method for preparing liposome suspensions as claimed in claim 1, wherein the injection apparatus in step (3) comprises at least one injection channel and a propulsion unit for controlling the flow rate.

3. The method for preparing liposome suspensions as claimed in claim 2, wherein the hole size of the at least one injection channel is less than 10 mm.

4. The method for preparing liposome suspensions as claimed in claim 2, wherein the propulsion unit is selected from the group consisting of pumps, gas propulsion unit, and other propulsion units.

5. The method for preparing liposome suspensions as claimed in claim 1, wherein the thermal condition is from 40° C. to 80° C.

6. The method for preparing liposome suspensions as claimed in claim 1, wherein the aqueous solution is an ion solution with ion concentration between 1 mM and 1 M.

7. The method for preparing liposome suspensions as claimed in claim 6, wherein the ion solution is selected from the group consisting of sodium chloride, polyacrylate, chondroitin sulfate A, polyvinylsulfate, phosphate, pyrophosphate, sulfate, citrate, tartarate, nitrilotiacetate, ethylenediamine tetraacetate, diethylenetriamine pentaacetate, and their salt derivatives thereof.

8. The method for preparing liposome suspensions as claimed in claim 1, wherein the volume ratio of the mixture to the aqueous solution is between 1:2 and 1:100.

9. The method for preparing liposome suspensions as claimed in claim 1, wherein the stirring speed and the aqueous solution is between 100 rpm and 500 rpm.

10. The method for preparing liposome suspensions as claimed in claim 1, wherein the flow rate of the injection apparatus in step (3) is between 10 mL/min and 1000 mL/min.

11. A method for preparing liposome suspensions as claimed in claims 1 to 10, further comprising an extrusion step for extruding the liposome suspensions, wherein the extrusion step comprises pressing the liposome suspensions through an extrusion unit comprising a filter membrane with pore size less than 100 nm.

12. The method for preparing liposome suspensions as claimed in claim 11, wherein the extrusion step comprises pressing the liposome suspensions through the extrusion unit comprising the filter membrane with pore size between 10 nm and 80 nm.

13. The method for preparing liposome suspensions as claimed in claim 11, wherein the pressure in the extrusion step is between 30 psi and 80 psi.

14. The method for preparing liposome suspensions as claimed in claim 11, wherein the extrusion flow rate in the extrusion step is between 2 L/min and 10 L/min.

15. A liposome suspension prepared by the method as claimed in claims 1 to 14, wherein an average particle size of liposomes of the liposome suspension is between 10 nm and 200 nm, and the particle size polydispersity index is between 0.01 and 0.5.

16. A method for encapsulating drug into liposomes of the liposome suspension as claimed in claim 15 comprising:
(1) providing a drug;
(2) dialyzing the liposome suspension to remove the alcoholic solvent; and
(3) mixing the drug and the liposome suspension, allowing the drug to be encapsulated in the liposomes.

17. The method for encapsulating drug into liposomes of the liposome suspension as claimed in claim 16, wherein the drug is selected from the group consisting of doxorubicin HCl, daunorubicin, gemcitabine, oxamniquine, fluconazole, itraconazole, ketoconazole, micronazole, irinotecan, and vinorelbine.

18. A liposome suspension comprising drug-encapsulated unilamellar vesicles produced by the method in claims 16 and 17, wherein an average particle size of the drug-encapsulated unilamellar vesicles is less than 200 nm.
